(19) United States
(12) Patent Application Publication
(10) Pub. No.: US 2026/0137614 A1
(43) Pub. Date: May 21, 2026

(54) ANHYDROUS COMPOSITION FOR MAKING UP KERATIN MATERIALS

(71) Applicant: L'OREAL, Paris (FR)

(72) Inventors: Zheng GONG, Shanghai (CN); Kun YU, Shanghai (CN)

(73) Assignee: L'OREAL, Paris (FR)

(21) Appl. No.: 19/120,462

(22) PCT Filed: Oct. 19, 2022
(86) PCT No.: PCT/CN2022/126155

§ 371 Date: Apr. 11, 2025

(51) Int. Cl. A61K 8/85; A61K 8/89; A61K 8/92; A61Q 1/06

(52) U.S. Cl. CPC A61K 8/85; A61K 8/89; A61K 8/92; A61Q 1/06; A61K 2800/31; A61K 2800/43; A61K 2800/805

(57) ABSTRACT

An anhydrous composition for making up keratin materials comprising 1) a polyester-containing mixture containing a) at least one polyester which is the reaction product of the following components: i) at least one polyglycerol-3; and ii) at least one dimer acid; and iii) at least one C8-C30 fatty monoacid, wherein the components i)-iii) reacted are in a mole ratio of 1 mole polyglycerol-3, 0.5 to 1 mol dimer acid, and from 0.1 to less than 2.0 mole fatty acids; and b) at least one non-volatile non-silicone oil H1; 2) at least one wax; 3) at least one colorant selected from titanium dioxides, iron oxides, organic pigments and colorants soluble in the medium of the composition; and optionally 4) at least one non-volatile non-silicone oil H2, identical or different from oil H1.

### TECHNICAL FIELD

The present invention relates to an anhydrous composition for making up keratin materials such as the skin and the lips, especially the lips. The present invention also relates to a process for making up keratin materials such as the skin and the lips, especially the lips.

### BACKGROUND

Compositions for making up the skin and/or the lips are produced to satisfy the need of wearing/non-transfer performance.

In general, when women use makeup products, especially lip products such as lipstick or lip gloss, they hope that the colour of this product is not easily transferred after application and that the product results in good sensory, for example, non-sticky and non-dry feeling.

For lip makeup products, especially the lipsticks or lip liquids with high amounts of pigments, usually their wearing/non-transfer performance is not satisfied by the consumers if the polymer with film-forming property is not included in the formula. Normally the most effective and widely used film formers are silicone-based polymers which require the large amount of silicone oil(s) to disperse. However, if the oil phase is non-silicone or contains a very low amount of silicone oil(s), there are very few non-silicone film formers available that can effectively enhance the wearing/non-transfer performance and also have the good compatibility with the oil phase.

Thus, there is still a need to obtain products for making up keratin materials such as the skin and the lips which provide a deposit having good colour transfer resistance and a good film-forming property.

### SUMMARY OF THE INVENTION

One object of the present invention is thus to provide products for making up keratin materials such as the skin and the lips which provide a deposit having good colour transfer resistance and a good film-forming property.

Another object of the present invention is to provide a process for making up keratin materials such as the skin and the lips, especially the lips.

Thus, according to one aspect, the present invention provides an solid anhydrous composition for making up keratin materials comprising:
1) a polyester-containing mixture containing
a) at least one polyester which is the reaction product of the following components:
i) at least one polyglycerol-3; and
ii) at least one dimer acid; and
iii) at least one C₈-C₃₀ fatty monoacid, wherein the components reacted are in a mole ratio of 1 mole polyglycerol-3, 0.5 to 1 mol dimer acid, and from 0.1 to less than 2.0 mole fatty acids; and
b) at least one non-volatile oil, preferably non-volatile non-silicone oil H1;
2) at least one wax;
3) at least one colorant selected from titanium dioxides, iron oxides, organic pigments and colorants soluble in the medium of the composition; and optionally
4) at least one non-volatile non-silicone oil H2, identical or different from oil H1.

The anhydrous composition according to the present invention is particularly useful for a lip product.

According to another aspect, the present invention provides a process for making up keratin materials such as the skin and the lips, especially the lips, comprising applying the anhydrous composition as described herein to the keratin materials.

### DETAILED DESCRIPTION OF THE INVENTION

Throughout the description, including the claims, the term “comprising a” should be understood as being synonymous with “comprising at least one”, unless otherwise mentioned. Moreover, the expression “at least one” used in the present description is equivalent to the expression “one or more”.

Throughout the description, including the claims, an embodiment defined with “comprising” or the like should be understood to encompass a preferable embodiment defined with “consisting substantially of” and a preferable embodiment defined with “consisting of”.

Other than in the operating examples, or where otherwise indicated, all numbers expressing quantities of components and/or reaction conditions are to be understood as being modified in all instances by the term “about,” with conventionally known meaning in the art, e.g., within 10% of the indicated number (e.g. “about 10%” means 9%-11% and “about 2%” means 1.8%-2.2%).

Throughout the description, including the claims, the “keratin material” according to the present invention is preferably skin, more preferably lip.

In the application, unless specifically mentioned otherwise, contents, parts and percentages are expressed on a weight basis.

Other characteristics and advantages of the invention will emerge more clearly on reading the description and the examples that follow.

The composition according to the present invention is in anhydrous form, which means the absence of water, or the presence of water in such an amount those skilled can determine it as free or substantially free of water. For example, a composition in anhydrous form according to the present invention may comprise 3% by weight or less, preferably 1% by weight or less of water, relative to the total weight of the composition. Preferably, a composition in anhydrous form according to the present invention does not comprise a detectable amount of water, wherein the “detectable amount” means an amount can be detected by a device conventionally used in the art to measure the water content.

### Component 1), Polyester-Containing Mixture

The anhydrous composition according to the present invention comprises a polyester-containing mixture as component 1). The polyester-containing mixture comprises a polyester and at least one non-volatile oil.

### Component a), Polyester

The polyester of the invention is the reaction product of the following components:
i) at least one polyglycerol-3;
ii) at least one dimer acid; and
iii) at least one C₈-C₃₀ fatty monoacid,
wherein the components reacted are in a mole ratio of 1 mole polyglycerol-3, 0.5 to 1 mol dimer acid, and 0.1 to less than 2.0 mole fatty acids.

Accordingly, the polyester of the invention can be designated as a “glycerin-based polyester”.

The term “polyglycerol-3” for the present invention means triglycerol alone or a mixture of polyglycerols comprising at least triglycerol, and the triglycerol preferably predominates in said mixture.

They are described as their synthesis in the patent applications US202110259945, US202110259946 and US202110259930.

According to a preferred embodiment, the polyester is a substantially or completely non-sequential reaction product.

By “substantially non-sequential reaction product” is meant the product is produced by substantially nonsequential reaction of the reacting components i)-iii).

By “substantially non-sequential reaction of the reacting components i)-iii)” is meant substantially the total content of each of the reactants i)-iii) to be reacted is added to the reaction vessel prior to commencing the reaction.

In one embodiment of the present disclosure, the total content of each of the reactants i)-iii) to be reacted is added to the reaction vessel prior to commencing the reaction, i.e., the reaction is completely non-sequential, and the polymer is a completely non-sequential reaction product of the components i)-iii). In other embodiments, at least 70-100% by weight, or 75-100% by weight, or 80-100% by weight, or 85-100% by weight, or 90-100% by weight, or 95-100% by weight, or 97-100% by weight of each of the reactants i)-iii) is added to the reaction vessel prior to commencing the reaction.

In one embodiment, the polyester is prepared by a one-step process that involves introducing all reactants to a reaction vessel and thereafter inducing a fully statistical addition of the dimer acid and the isostearic acid to the polyglycerol.

### Component i), Polyglycerol-3

For the present invention, polyglycerol-3 means triglycerol alone or a mixture of polyglycerols comprising at least triglycerol, and the triglycerol preferably predominates in said mixture. The triglycerol has the formula H—[—OGly]₃-OH wherein Gly is the residue of a molecule of glycerol.

A polyglycerol-3 according to the invention in the form of mixture of polyglycerols comprising triglycerol may contain polyglycerols which can be any oligocondensation products of glycerol and having the formula (I):

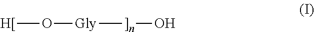

wherein each Gly is independently the residue of a molecule of glycerol after removal of two hydroxyl groups; and n is an average of from 2 to 10.

Generally, most of the groups Gly will be of the formula: —CH₂—CHOH—CH₂—, although residues comprising etherification at the secondary or even tertiary hydroxyl groups are considered to be within the scope of “Gly” and, thus, may also be present. Examples of oligoglycerols include diglycerol, triglycerol, tetraglycerol, pentaglycerol, hexaglycerol, heptaglycerol, octaglycerol, nonaglycerol, decaglycerol, and mixtures of these. Particularly useful polyglycerols are those of the formula (I) wherein n is particularly from 2 to 7, more particularly from 2 to 5 and especially 2, 3 or 4, or mixtures of oligoglycerols in these ranges.

Particularly suitable examples of polyglycerol-3 comprise a mixture of oligoglycerols having the following oligomer distribution wherein all weight percentages are based on a total content of the polyglycerols.
Glycerol: 0 to 30% by weight, preferably 0 to 20% by weight, more preferably 0 to 15% by weight;
Diglycerol: 10 to 40% by weight, preferably 15 to 35% by weight, more preferably 20 to 32% by weight;
Triglycerol: 10 to 65% by weight, preferably 15 to 60% by weight, more preferably 18 to 55% by weight;
Tetraglycerol: 2 to 25% by weight, preferably 5 to 20% by weight, more preferably 8 to 20% by weight;
Pentaglycerol: 0 to 15% by weight, preferably 0 to 10% by weight, more preferably 0 to 5% by weight;
Hexaglycerol: 0 to 15% by weight, preferably 0 to 10% by weight, more preferably 0 to 5% by weight;
Heptaglycerol: 0 to 10% by weight, preferably 0 to 5% by weight, more preferably 0 to 3% by weight;
Octaglycerol: 0 to 10% by weight, preferably 0 to 5% by weight, more preferably 0 to 3% by weight;
Nonaglycerol: 0 to 5% by weight, preferably 0 to 3% by weight, more preferably 0 to 2% by weight;
Decaglycerol: 0 to 5% by weight, preferably 0 to 3% by weight, more preferably 0 to 2% by weight relative to the total weight of the polyglycerol-3 under the form of a mixture.
   In one embodiment, a polyglycerol-3 under the form of mixture comprises the following oligomer distribution:
Glycerol: 0 to 30% by weight
Diglycerol: 15 to 40% by weight
Triglycerol: 10 to 55% by weight
Tetraglycerol: 2 to 25% by weight
Pentaglycerol and higher components: 0 to 15% by weight relative to the total weight of the polyglycerol-3 under the form of a mixture.

In one embodiment, a polyglycerol-3 under the form of mixture comprises at least 40% by weight, or at least 45% by weight, or at least 50% by weight, based on a total weight of the polyglycerol-3 under the form of mixture, of a combination of diglycerol and triglycerol.

In one embodiment, a polyglycerol-3 under the form of mixture comprises at least 20% by weight, or at least 25% by weight of diglycerol; at least 15% by weight, or at least 18% by weight of triglycerol; at least 10% by weight, or at least 12% by weight of tetraglycerol; wherein all weight percentages are based on a total content of the of the polyglycerol-3 under the form of mixture.

A particularly preferred polyglycerol-3 comprises at least 25% by weight diglycerol, at least 45% by weight triglycerol, and at least 10% by weight tetraglycerol, based on a total content of the of the polyglycerol-3 under the form of mixture s.

Analysis of any such polyglycerol-3 in the form of mixture of polyglycerols according to the invention can be done to determine it's median, mean, or “average” polyglycerol number. Oligoglycerol examples above with both narrow and broad distributions can be equally designated as “polyglycerol-3”, as this is the closest integer to the mean and/or median.

Component ii), Dimer Acid

The dimer acid useful for the polyester can be any dicarboxylic acid having at least 4 carbon atoms. They can be straight-chain or branched, such as, for example, dimers prepared from malonic acid, succinic acid, fumaric acid, dimethylglutaric acid or trimethyladipic acid, and their anhydrides.

Dimer fatty acids are especially useful. As is known, these are mixtures of acyclic and cyclic dicarboxylic acids which are obtained by a catalyzed dimerization reaction of unsaturated fatty acids having 12 to 22 carbon atoms.

For the preparation and use of dimer acids and their physical and chemical properties, reference is made to the publication “The Dimer Acids: The chemical and physical properties, reactions and applications”, Ed. E. C. Leonard; Humko Sheffield Chemical, 1975, Memphis, Tenn.

The dicarboxylic acids can also contain, to a lesser extent, tri- and polyfunctional carboxylic acids. The functionality of the mixture should not exceed a value of 2.4 molar average.

Preferred are dimer acids typically derived from triglycerides rich in C₁₈ ester groups, which can be hydrolyzed to produce C₁₈ unsaturated monoacidic fatty acids. The raw materials can be derived from tall oil and rapeseed oil, but other natural sources including flaxseed, soybean, pumpkin, walnut can be used. The target monoacids used in the reaction are rich in oleic and linoleic acid forms described in the fatty acid listing contained below. Dimerization leads primarily to the dimerization of unsaturated fatty acids, however trimers are also formed. After reaction, the product can be kept as a mixture of reaction products or it can be further distilled or otherwise separated into molecular weight fractions. In one embodiment, the dimerization reaction produces a majority (at least 60 wt % by weight, more preferably at least 75 wt % by weight) of dimer acid (C₃₆ diacid) but also produces C₅₄ trimer acids (less than 30 wt % by weight, more preferably less than 25% by weight).

In one case, commercially available standard dimer acid from Croda, Pripol 1025®, is used which contains 72 wt % by weight dimer and 19 wt % by weight trimer acid.

In another case, hydrogenated standard dimer acid from Oleon, Radiacid 0960, is used which contains 87% by weight dimer, and 10% by weight trimer acid. In both cases, the polymer as described is characterized by higher molecular weight, more hydrophobic character, and higher viscosity than can be provided by pure, lower molecular weight, diacids. The presence of trimer acid further enhances the molecular weight and performance of these polymers.

In one embodiment, the polyester of the present invention is prepared from at least one hydrogenated dimer acid.

In another embodiment, the polyester is prepared from a hydrogenated dimer acid comprising hydrogenated dimerized C₁₈ fatty acids, which hydrogenated dimer acid is obtained through dimerization of unsaturated C₁₈ fatty acids and subsequent hydrogenation.

In one embodiment, the hydrogenated dimer acid contains a trimer acid content ranging from about 5-25% by weight, based on a total weight of hydrogenated dimer acid.

In another embodiment, the hydrogenated dimer acid contains a majority (at least 60% by weight, more preferably at least 75% by weight, but n t more than 95% by weight, or better not more than 90% by weight, or better still not more than 85% by weight) of hydrogenated dimer acid (C₃₆ diacid) and also contains C₅₄ hydrogenated trimer acids (less than 30% by weight, more preferably less than 25% by weight, but greater than 5% by weight, more preferably greater than 10% by weight).

Component iii), C₈-C₃₀ Fatty Monoacids

C₈-C₃₀ fatty monoacids useful for the polyester can include naturally occurring or refined fatty acids, such as hydrolyzed rapeseed oil, sunflower oils etc, however these contain both lower and higher MW chains. Useful fatty monoacids can be linear, branched, saturated, unsaturated, and aromatic materials with acidity provided by carboxylic acid moieties.

Useful acids include Caprylic acid (C₈), Pelargonic acid (C₉), Capric acid (C₁₀), Undecylic acid (C₁₁), Lauric acid (C₁₂), Tridecylic acid (C₁₃), Myristic acid (C₁₄), Pentadecylic acid (C₁₅), Palmitic acid (C₁₆), Margaric acid (C₁₇), Stearic acid (C₁₈), Isostearic acid (C₁₈), Nonadecylic acid (C₁₉), Arachidic acid (C₂₀), Behenic acid (C₂₂), and Lignoceric acid (C₂₄).

Comparing stearic and isostearic acid shows that branching leads to high melting point and results in a low viscosity at room temperature for isostearic acid, vs a solid material for stearic acid. This lower viscosity can be helpful in materials handling of raw materials and also in enabling esters made with this acid to retain liquid properties. Branched-chain fatty acids often contain a single methyl branch along the linear carbon chain and are produced in nature through microbial action. Isotearic acid is available as a reaction by product in the creation of dimer acid described above.

Another route to obtaining a liquid product is to use unsaturated linear and branched fatty monoacids. These unsaturated acids can include Palmitoleic acid (C16:1), Vaccenic acid (C18:1), Oleic acid (C18:1), Elaidic acid (C18:1), Linoleic acid (C18:2), Linolelaidic acid (C18:2), α-Linolenic acid (C18:3), γ-Linolenic acid (C18:3), Stearidonic acid (C18:4), Paullinic acid (C20:1), Gondoic acid (C20:1), Dihomo-γ-linolenic acid (C20:3), Mead acid (C20:3), Arachidonic acid (C20:4), Eicosapentaenoic acid (C20:5), Erucic acid (C22:1), Docosatetraenoic acid (C22:4), Cervonic acid (C22:6), and Nervonic acid (C24:1). As is well-known to persons skilled in the art, the designation means the length of the carbon chain is X carbon atoms; and there are Y number of double bonds in the chain.

In one embodiment, isostearic acid will be preferred.

In an especially preferred embodiment, the polyester of the invention is a substantially or totally non sequential reaction product of the following components:
i) at least one polyglycerol-3 under the form of mixture comprising at least 25% by weight diglycerol, at least 45% by weight triglycerol, and at least 10% by weight tetraglycerol, in each case based on a total weight of polyglycerol-3 under the form of mixture;
ii) at least one hydrogenated dimer acid containing at least 60% by weight of hydrogenated C₃₆ diacid and 5-25% by weight of hydrogenated C₅₄ triacid, in each case based on a total weight of hydrogenated acid; and
iii) isostearic acid.

In one embodiment, the polyester is prepared by a one-step process that involves introducing all reactants to a reaction vessel and thereafter inducing a fully statistical addition of the dimer acid and the isostearic acid to the polyglycerol.

In one embodiment, it is preferable to have a total degree of esterification of available polyglycerol hydroxyl moieties (total esterification) of from 24% to 74% and a degree of esterification of available polyglycerol hydroxyl moieties by dimer acid alone (esterification with dimer acid) of 20% to 40%. More importantly, the degree of esterification by the end-cap units (esterification with monoacid) are also set in this disclosure and it is important to maintain the esterification with monoacid from 4% to 40%.

It is more preferable to have a total esterification of 28% to 57%, including an esterification with dimer acid of 20% to 30% and an esterification with monoacid between 8% and 27%.

It is still more preferable to have a total esterification of 33% to 48%, including an esterification with dimer acid of 20% to 28% and an esterification with monoacid between 13% and 20%.

It is still more preferable to have a total esterification of 24% to 74%, including an esterification with hydrogenated dimer acid of 20% to 40% and an esterification with monoacid between 4% and 40%.

It is still more preferable to have a total esterification of 28% to 57%, including an esterification with hydrogenated dimer acid of 20% to 30% and an esterification with monoacid between 8% and 27%.

It is also still more preferable to have a total esterification of around 40%, including an esterification with hydrogenated dimer acid of around 20% and an esterification with monoacid of around 20%.

It is also still more preferable to have a total esterification of around 40%, including an esterification with hydrogenated dimer acid of around 27% and an esterification with monoacid of around 13%.

In one embodiment, the components reacted are in a mole ratio of 1 mole polyglycerol-3, 0.5 to 1 mol dimer acid and 0.2 to 1.7 mole fatty acid.

In another embodiment, the components reacted are in a mole ratio of 1 mole polyglycerol-3, 0.5 to 0.75 mol dimer acid, and 0.4 to 1.35 mole isostearic acid.

In another embodiment, the components reacted are in a mole ratio of 1 mole polyglycerol-3, 0.5 to 0.7 mol dimer acid, and 0.65 to 1 mole isostearic acid.

In another embodiment, the components reacted are in a mole ratio of 1 mole polyglycerol-3, 0.5 to 1 mol hydrogenated dimer acid, and 0.2 to 1.7 mole isostearic acid.

In another embodiment, the components reacted are in a mole ratio of 1 mole polyglycerol-3, 0.5 to 0.75 mol hydrogenated dimer acid, and 0.4 to 1.35 mole isostearic acid.

In another embodiment, the components reacted are in a mole ratio of 1 mole polyglycerol-3, 0.5 to 0.7 mol hydrogenated dimer acid, and 0.65 to 1 mole isostearic acid.

In another embodiment, the components reacted are in a mole ratio of 1 mole polyglycerol-3, 0.67 mole hydrogenated C₃₆ dimer acid, and 0.67 mole isostearic acid.

In one more preferred embodiment, the components reacted are in a mole ratio of 1 mole polyglycerol-3, 0.5 mole hydrogenated C₃₆ dimer acid, and 1 mole isostearic acid.

By adjusting the fatty acid end-cap mole ratio and balancing the amount of polyglycerol and dimer acid, it is also possible to control the degree of dimer acid-polyglycerol-extension and end-capping so that cross-linking, for example, via trimer acid, leads to much higher viscosities.

The target viscosity of the pure polymer should be >50,000 cPs and less than 5,000,000 mPa·s at 25° C.

In one preferred embodiment, the target viscosity is >75,000 mPa·s and <2,500,000 mPa·s at 25° C.

In another preferred embodiment, the target viscosity is >100,000 mPa·s and <2,000,000 mPa·s at 25° C.

In a more preferred embodiment, the target viscosity is >1,000,000 mPa·s and <2,000,000 mPa·s at 25° C.

The viscosity is measured using a MCR302 Rheometer® from Anton Paar Inc. Roughened or smooth 50 mm diameter twin flat plates were used, covered with polymer sample, adjusted to a gap of 0.5 to 1 mm, and both temperature an shear rate sweeps performed. The polyesters of the invention display Newtonian behavior and thus have a constant viscosity over a wide range of shear rates. Also, the polyesters of this disclosure demonstrate a reduced viscosity with temperature. Thus, measures of viscosity are reported at a precisely controlled temperature and typically as a shear-rate of 1 The values are reported in unit of mPa·s

The polyesters of the invention are characterized by weight-average molecular weights >2500 Da and <1,000,000 Da measured with GPC using linear polystyrene standards.

The GPC column used for these tests consists of: Phenolgel, 300×4.6 mm; a continuous phase of Tetrahydrofuran (THF) being used and injected at 0.35 mL/min, with the column oven held at 40° C.; a 50 μL injection, and Wyatt refractive index Ri detector. The calibration standards used are strictly linear polystyrene made to be mono-dispersed. Narrow-range polystyrene GPC calibration standards are prepared in mobile phase and have peak molecular weights of 1,290,000 Da; 560,000 Da; 65,500 Da; 28,500 Da; 10,100 Da; 1,680 Da; 580 Da and 208 Da. From standard methodologies, the weight-average and number-average molecular weight are automatically calculated by standard GPC software.

In a preferred embodiment, the disclosed polyesters have a weight-average molecular weight >4000 Da and <250,000 Da measured with GPC using linear polystyrene standards. In a more preferred embodiment, the disclosed polymers have a weight-average molecular weight >5000 Da and <150,000 Da measured with GPC using linear polystyrene standards.

In yet another embodiment, the polyester of the invention exhibits a combination of weight-average molecular weight >5000 Da and <150,000 Da measured with GPC using linear polystyrene standards and a viscosity at 25° C. >100,000 mPa·s and <2,000,000 mPa·s.

In a preferred embodiment, the polyester of the invention is a substantially or completely non sequential reaction product of the following components:
i) at least one polyglycerol-3 under the form of mixture comprising at least 25% by weight diglycerol, at least 45% by weight triglycerol, and at least 10% by weight tetraglycerol, in each case based on a total weight of polyglycerol-3 under the form of mixture;
ii) at least one hydrogenated dimer acid containing at least 60% by weight of hydrogenated C₃₆ diacid and 5-25% by weight of hydrogenated C₅₄ triacid, in each case based on a total weight of hydrogenated acid; and
iii) isostearic acid;
wherein the polyester exhibits a combination of weight-average molecular weight >5000 Da and <15,000 Da measured with GPC using linear polystyrene standards and viscosity >100,000 mPa·s and <2,000,000 mPa·s at 25° C.; and wherein the polyester is also characterized by a total esterification of about 40%, including an esterification with hydrogenated dimer acid of about 27%, and an esterification with monoacid of about 13%.

In practice, since the raw ingredients contain a range of polyglycerol-3 units and a range of dimer and trimer acid content, the above numbers can be adjusted using the actual (and non-theoretical) hydroxyl moieties and carboxylic acid moieties as they are determined by standard methods such as mass spectrometry, NMR, and liquid chromatography. The above esterification ranges are based on the idealized structure of polyglycerol-3 and C₃₆-dimer acid. Actual ranges may thus be slightly different than the values given above and can be calculated based on these analytical analyses.

It is more practical to define the extent of polymerization by the final acid value. The initial acid values, in light of the distribution of polyglycerol, monoacid, and polyacid moieties present, can be reliably calculated using the actual acid value determined by the raw ingredient used.

For one example, the initial total Acid Value (“AV” which is commonly defined as mg KOH/g total reactant) is 135 AV. This includes 68 AV for dimer acid and 67 AV for isostearic acid of one preferred embodiment containing 1 mole polyglycerol-3, 0.5 mole hydrogenated C₃₆ dimer acid, and 1 mole isostearic acid. All preferred ratio embodiments described above have corresponding initial AV that can be calculated. When during course of the polymerization reaction, the AV units are reduced, this ratio gives the percent conversion of the reaction from total initial reactive acid moieties to final residual acid moieties. Thus, the completion of reaction is the 1 minus the ratio of final AV to initial AV.

In one embodiment, the polyesters of the invention have final acid values of 0.1 to <25 mg KOH/g polymer.

In a preferred embodiment, the polyesters of the invention have final acid values 0.1 to <10 mg KOH/g polymer.

In a more preferred embodiment, the polyesters of the invention have final acid values 0.1 to <5 mg KOH/g polymer.

Expressing completion of reaction as (1-Final AV)/Initial AV, the completion of reaction of such reactor mixtures to final polymer is >80%.

In a preferred embodiment, the completion of reaction of such reactor mixtures to final polymer is >90%.

In a more preferred embodiment, the completion of reaction of such reactor mixtures to final polymer is >95%.

In a more preferred embodiment, the polyester of the invention is the reaction product of a polyglycerol-3, a C₃₆ hydrogenated dimer acid and isostearic acid in a molar ratio of 1/0.5/1 as disclosed in example 10 of US 2021/0259945.

### Component b), Non-Volatile Oil

Non-volatile oils are useful as component b) to form the polyester-containing mixture with the polyester of component a).

Solution of Polyester with an Non-Volatile Oil

According to a particular preferred embodiment of the invention, the polyester-containing mixture may be in the form of an oily solution containing the polyester as component a) and b) at least one non-volatile oil.

This type of solution of oil(s) and polyester is described in patent applications US202110259945, US202110259946 and US202110259930.

### Non-Volatile Oils

The oil which is present in the mixture of the invention may be chosen from the group constituted of hydrocarbon-based oils, silicone oils and mixtures thereof.

The term “oil” refers to a fatty substance that is liquid at room temperature (25° C.) and atmospheric pressure (760 mmHg, i.e. 105 Pa).

For the purposes of the present invention, the term “silicone oil” refers to an oil comprising at least one Si—O group, and more particularly an organopolysiloxane.

The term “hydrocarbon-based oil” refers to an oil mainly containing hydrogen and carbon atoms and possibly one or more functions chosen from hydroxyl, ester, ether and carboxylic functions. That is, the hydrocarbon-based oil is preferably a non-silicone oil. The term “non-volatile oil” means an oil remaining on the skin or the keratin fiber at ambient temperature and atmospheric pressure for at least several hours and having in particular a vapor pressure of less than 2.66 Pa, preferably less than 0.13 Pa. By way of example, the vapor pressure can be measured according to the static method or by the effusion method by isothermal thermogravimetry, depending on the vapor pressure (OECD standard 104).

According to the invention, for the purpose of use as component b), a non-volatile non-silicone oil is preferred. As examples of non-volatile non-silicone oils that may be used in the invention, mention may be made of:
vegetable oils, such as phytostearyl esters, for instance phytostearyl oleate, phytostearyl isostearate and lauroyl/octyldodecyl/phytostearyl glutamate (AJINOMOTO, ELDEW PS203), diesters such as diisopropyl sebacate, triglycerides constituted of fatty acid esters of glycerol, in particular in which the fatty acids may have chain lengths ranging from C4 to C36, and in particular from C18 to C36, it being possible for these oils to be linear or branched, and saturated or unsaturated; these oils may in particular be heptanoic or octanoic triglycerides, shea oil, alfalfa oil, poppy seed oil, pumpkin oil, millet oil, barley oil, quinoa oil, rye oil, candlenut oil, passionflower oil, aloe oil, sweet almond oil, peach kernel oil, groundnut oil, argan oil, avocado oil, baobab oil, barrage oil, broccoli oil, calendula oil, camelina oil, canola oil, carrot oil, safflower oil, hemp oil, rapeseed oil, cotton seed oil, coconut oil, marrow seed oil, wheat germ oil, jojoba oil, lily oil, macadamia oil, corn oil, meadowfoam oil, St. John's Wort oil, monoi oil, hazelnut oil, apricot kernel oil, nut oil, olive oil, evening primrose oil, palm oil, blackcurrant seed oil, kiwi seed oil, grape seed oil, pistachio oil, pumpkin oil, winter squash oil, quinoa oil, musk rose oil, sesame oil, soya oil, sunflower oil, castor oil and watermelon oil, and mixtures thereof, or alternatively caprylic/capric acid triglycerides, for instance those sold by the StEarineries Dubois company or those sold under the names Miglyol 810®, 812® and 818® by the Dynamit Nobel company;
linear or branched hydrocarbons, of mineral or synthetic origin, such as liquid paraffins and derivatives thereof as C₁₃-C₁₆ isoparaffins, petroleum jelly, polydecenes, polybutenes, hydrogenated polyisobutene such as Parleam, or squalane;
synthetic ethers containing from 10 to 40 carbon atoms, such as dicaprylyl ether;
synthetic esters, e.g., esters of C₄-C₂₂ dicarboxylic or tricarboxylic acids and of C₁-C₂₂ alcohols and esters of mono-, di- or tri-carboxylic acids and of C₂-C₂₆ di-, tri-, tetra- or penta-hydroxy alcohols, examples thereof comprising diethyl sebacate; diisopropyl sebacate; diisopropyl adipate; di-n-propyl adipate; dioctyl adipate; diisostearyl adipate; dioctyl maleate; glyceryl undecylenate; octyldodecyl stearoyl stearate; pentaerythrityl monoricinoleate; pentaerythrityl tetraisononanoate; pentaerythrityl tetrapelargonate; pentaerythrityl tetraisostearate; pentaerythrityl tetraoctanoate; propylene glycol dicaprylate; propylene glycol dicaprate; tridecyl erucate; triisopropyl citrate; triisostearyl citrate; glyceryl trilactate; glyceryl trioctanoate; trioctyldodecyl citrate; trioleyl citrate; propylene glycol dioctanoate; neopentyl glycol diheptanoate; diethylene glycol diisononanoate; ethylene glycol distearate; diethylene glycol distearate and polyethylene glycol distearate;
copolymers of polyols and of diacid dimers, and esters thereof, such as Hailuscent ISDA, or the copolymer of dilinoleic acid/butanediol,
fatty alcohols that are liquid at room temperature, containing a branched and/or unsaturated carbon chain containing from 12 to 26 carbon atoms, for instance octyldodecanol, isostearyl alcohol, 2-butyloctanol, 2-hexyldecanol, 2-undecylpentadecanol or oleyl alcohol;
higher fatty acids, such as oleic acid, linoleic acid or linolenic acid;
carbonates, such as dicaprylyl carbonate;
acetates;
citrates;
optionally partially hydrocarbon-based and/or silicone fluoro oils, for instance fluoro silicone oils, fluoropolyethers and fluorosilicones as described in EP-A-847 752;
polydimethylsiloxanes (INCI name: Dimethicone) (all the radicals R1 to R6 and X represent methyl), in particular with a viscosity from 50 to 500 cSt, especially 350 cSt, such as the commercial products sold under the names Belsil DM 350® from the company Wacker, and Xiameter PMX-200 Silicone Fluid® 350 CS from the company Dow Corning, and more particularly the polydimethylsiloxanes (INCI name: Dimethicone) with a viscosity from 50 to 150 cSt, especially 100 cSt, such as the commercial products sold under the names Belsil DM 100® from the company Wacker, and Xiameter PMX-200 Silicone Fluid 100 CS® from the company Dow Corning.
phenyl silicones, for instance phenyl trimethicones, phenyl dimethicones, phenyltrimethylsiloxydiphenylsiloxanes, diphenyl dimethicones, diphenylmethyldiphenyltrisiloxanes and 2-phenylethyl trimethyl-siloxysilicates; and
mixtures thereof.

According to a preferred embodiment, the oil will be selected from hydrocarbon-based non-volatile oils and more preferably selected from fatty acid triglycerides containing from 4 to 24 carbon atoms, for instance caprylic/capric acid triglycerides.

According to a preferred embodiment, the composition according to the present invention comprises one non-volatile hydrocarbon-based oil chosen from polybutenes, polyisobutenes, hydrogenated polyisobutenes, polydecenes hydrogenated polydecenes, squalene, esters of C₄-C₂₂ dicarboxylic or tricarboxylic acids and of C₁-C₂₂ alcohols and esters of mono-, di- or tri-carboxylic acids and of C₂-C₂₆ di-, tri-, tetra- or penta-hydroxy alcohols, and mixtures thereof.

The oily solution of polyester of the invention may be obtained by mixing the polyester with the organic at around 80-100° C. The combination is then further cooled to 50-70° C. for discharging from the reactor and placement into storage.

The oily solution of polyester of the invention contains preferably the polyester in a concentration of from 10 to 99% by weight, more preferably from 30 to 90% by weight, more particularly 50 to 80% by weight related to the total weight of the mixture.

According to an embodiment of the invention, a non-volatile non-silicone described here can be used as oil H1, i.e., component b), to form the polyester-containing mixture of component 1) with the polyester of component a), e.g., by forming an oily solution described here. In addition, a non-volatile non-silicone described here can be used as oil H2, i.e., component 4), identical or different from oil H1, to formulate the anhydrous composition with the components 1), 2) and 3).

According to a particular preferred embodiment, the anhydrous composition of the invention contains an oily solution containing:
a) a polyester obtained by the reaction of
i) at least a polyglycerol-3, and
ii) a C₃₆ hydrogenated acid dimer; and
iii) isostearic acid; wherein the components reacted are in a mole ratio of 1 mole polyglycerol-3, 0.5 to 1 mol hydrogenated dimer acid, and 0.1 to less than 2.0 mole fatty acids; and
b) a caprylic/capric triglyceride having as INCI name: DIISOSTEAROYL POLYGLYCERYL-3 DIMER DILINOLEATE (AND) CAPRYLIC/CAPRIC TRIGLYCERIDE.

Such oily solution is commercialized under the name SOLAMAZE NATURAL® by the company NOURYON comprising 60% by weight in active material of polyester.

The polyester-containing mixture of component 1) may be present in the anhydrous composition in an amount ranging from 0.01% to 20% by weight, more preferably from 1% to 10% by weight, even more preferably from 1% to 5% by weight, based on the total weight of the total anhydrous composition.

### Component 2), Wax(s)

The anhydrous composition according to the present invention comprises a wax as component 2).

The wax under consideration in the context of the present invention is generally a lipophilic compound that is solid at room temperature (25° C.), with a solid/liquid reversible change of state, having a melting point of greater than or equal to 30° C., preferably greater than or equal to 40° C., which may be up to 200° C. and in particular up to 120° C.

Waxes used in the present invention includes waxes of animal origin, waxes of plant origin, waxes of mineral origin, synthetic waxes, and various fractions of waxes of natural origin.

Animal waxes include, but are not limited to, beeswax, spermaceti, lanolin wax, derivatives of lanoline and China insect waxes. Vegetable waxes includes, but are not limited to, rice wax, carnauba wax, candelilla wax, ouricurry wax, cork fiber wax, sugar cane wax, cocoa butter, Japan wax and sumac wax. Mineral waxes include, but are not limited to, montan wax, microcrystalline waxes, paraffins, ozokerite, petroleum jelly and ceresine. Synthetic waxes include, but are not limited to, polyethylene homopolymer and compolymer waxes, synthecit beewax, waxes obtained by the Fisher and Tropsch synthesis, and silicon waxes.

Despite the origin, the useful wax may be hydrocarbon-based waxes, for instance beeswax, lanolin wax or Chinese insect wax; rice wax, carnauba wax, candelilla wax, ouricury wax, esparto grass wax, cork fibre wax, sugar cane wax, Japan wax and sumach wax, Helianthus annuus (sunflower) seed wax; montan wax, microcrystalline waxes, paraffins and ozokerite; polyethylene waxes, polymethylene waxes, the waxes obtained by Fisher-Tropsch synthesis and waxy copolymers, and also esters thereof.

Waxes obtained by catalytic hydrogenation of animal or vegetable oils, having linear or branched C₈-C₃₂ fatty chains are also used, as well as fatty esters and glycerides.

According to a particularly preferred embodiment, the wax(es) used in the present invention may be chosen from polyethylene, synthetic wax, paraffin, microcrystalline wax, or mixtures thereof.

The wax(es) can be present in an amount ranging 0.01% to 20% by weight, preferably 0.1% to 15% by weight, more preferably 0.5% to 10% by weight, relative to the total weight of the anhydrous composition.

### Component 3), Colorant(s)

For the purposes of the present invention, the term “colorant” means a compound that is capable of producing a colored optical effect when it is formulated in sufficient amount in a suitable cosmetic medium.

The colorant under consideration in the context of the present invention may be chosen from water-soluble or water-insoluble, liposoluble (in particular colorant soluble in the medium of the composition) or non-liposoluble, organic or inorganic colorants, and materials with an optical effect, and mixtures thereof.

### Water-Soluble Dyes

The colorants useful according to the present invention can comprise water-soluble dyes. For the purposes of the present invention, the term “water-soluble dye” means any natural or synthetic, generally organic compound which is soluble in an aqueous phase or water-miscible solvents and which is capable of imparting colour. In particular, the term “water-soluble” is intended to characterize the capacity of a compound to dissolve in water, measured at 25° C., to a concentration at least equal to 0.1 g/l (production of a macroscopically isotropic, transparent, coloured or colourless solution). This solubility is in particular greater than or equal to 1 g/l.

As water-soluble dyes that are suitable for use in the present invention, mention may be made in particular of synthetic or natural water-soluble dyes, for instance FD&C Red 4 (CI: 14700), DC Red 6 (Lithol Rubine Na; CI: 15850), DC Red 22 (CI: 45380), DC Red 28 (CI: 45410 Na salt), DC Red 30 (CI: 73360), DC Red 33 (CI: 17200), DC Orange 4 (CI: 15510), FDC Yellow 5 (CI: 19140), FDC Yellow 6 (CI: 15985), DC Yellow 8 (CI: 45350 Na salt), FDC Green 3 (CI: 42053), DC Green 5 (CI: 61570), FDC Blue 1 (CI: 42090).

As non-limiting illustrations of sources of water-soluble colorant(s) that may be used in the context of the present invention, mention may be made in particular of those of natural origin, such as extracts of cochineal carmine, of beetroot, of grape, of carrot, of tomato, of annatto, of paprika, of henna, of caramel and of curcumin.

Thus, the water-soluble colorants that are suitable for use in the present invention are in particular carminic acid, betanin, anthocyans, enocyanins, lycopene, β-carotene, bixin, norbixin, capsanthin, capsorubin, flavoxanthin, lutein, cryptoxanthin, rubixanthin, violaxanthin, riboflavin, rhodoxanthin, cantaxanthin and chlorophyll, and mixtures thereof. They may also be copper sulfate, iron sulfate, water-soluble sulfopolyesters, rhodamine, betaine, methylene blue, the disodium salt of tartrazine and the disodium salt of fuchsin.

Some of these water-soluble colorants are in particular approved for food use. Representatives of these dyes that may be mentioned more particularly include dyes of the carotenoid family, referenced under the food codes E120, E162, E163, E160a-g, E150a, E101, E100, E140 and E141.

### Pigments

The colorants useful according to the present invention can comprise pigments, including inorganic pigments and organic pigments.

The term “pigments” should be understood as meaning white or coloured, inorganic (mineral) or organic particles, which are insoluble in a liquid organic phase, and which are intended to color and/or opacify the composition and/or the deposit produced with the composition.

The pigments may be chosen from mineral pigments, organic pigments and composite pigments (i.e. pigments based on mineral and/or organic materials).

The pigments may be chosen from monochromatic pigments, lakes and pigments with an optical effect, for instance goniochromatic pigments and nacres.

The mineral pigments may be chosen from metal oxide pigments, chromium oxides, iron oxides (black, yellow, red), titanium dioxide, zinc oxides, cerium oxides, zirconium oxides, chromium hydrate, manganese violet, Prussian blue, ultramarine blue, ferric blue, metal powders such as aluminium powders and copper powder, and mixtures thereof.

Organic lakes are organic pigments formed from a dye attached to a substrate.

The lakes, which are also known as organic pigments, may be chosen from the materials below, and mixtures thereof:
cochineal carmine;
organic pigments of azo dyes, anthraquinone dyes, indigoid dyes, xanthene dyes, pyrene dyes, quinoline dyes, triphenylmethane dyes or fluorane dyes.

Among the organic pigments that may in particular be mentioned are those known under the following names: D&C Blue No. 4, D&C Brown No. 1, D&C Green No. 5, D&C Green No. 6, D&C Orange No. 4, D&C Orange No. 5, D&C Orange No. 10, D&C Orange No. 11, D&C Red No. 6, D&C Red No. 7, D&C Red No. 17, D&C Red No. 21, D&C Red No. 22, D&C Red No. 27, D&C Red No. 28, D&C Red No. 30, D&C Red No. 31, D&C Red No. 33, D&C Red No. 34, D&C Red No. 36, D&C Violet No. 2, D&C Yellow No. 7, D&C Yellow No. 8, D&C Yellow No. 10, D&C Yellow No. 11, FD&C Blue No. 1, FD&C Green No. 3, FD&C Red No. 40, FD&C Yellow No. 5, FD&C Yellow No. 6;
the organic lakes may be insoluble sodium, potassium, calcium, barium, aluminium, zirconium, strontium or titanium salts of acidic dyes such as azo, anthraquinone, indigoid, xanthene, pyrene, quinoline, triphenylmethane or fluorane dyes, these dyes possibly comprising at least one carboxylic or sulfonic acid group.

The organic lakes may also be supported on an organic support such as rosin or aluminium benzoate, for example.

Among the organic lakes, mention may be made in particular of those known under the following names: D&C Red No. 2 Aluminium lake, D&C Red No. 3 Aluminium lake, D&C Red No. 4 Aluminium lake, D&C Red No. 6 Aluminium lake, D&C Red No. 6 Barium lake, D&C Red No. 6 Barium/Strontium lake, D&C Red No. 6 Strontium lake, D&C Red No. 6 Potassium lake, D&C Red No. 7 Aluminium lake, D&C Red No. 7 Barium lake, D&C Red No. 7 Calcium lake, D&C Red No. 7 Calcium/Strontium lake, D&C Red No. 7 Zirconium lake, D&C Red No. 8 Sodium lake, D&C Red No. 9 Aluminium lake, D&C Red No. 9 Barium lake, D&C Red No. 9 Barium/Strontium lake, D&C Red No. 9 Zirconium lake, D&C Red No. 10 Sodium lake, D&C Red No. 19 Aluminium lake, D&C Red No. 19 Barium lake, D&C Red No. 19 Zirconium lake, D&C Red No. 21 Aluminium lake, D&C Red No. 21 Zirconium lake, D&C Red No. 22 Aluminium lake, D&C Red No. 27 Aluminium lake, D&C Red No. 27 Aluminium/Titanium/Zirconium lake, D&C Red No. 27 Barium lake, D&C Red No. 27 Calcium lake, D&C Red No. 27 Zirconium lake, D&C Red No. 28 Aluminium lake, D&C Red No. 30 lake, D&C Red No. 31 Calcium lake, D&C Red No. 33 Aluminium lake, D&C Red No. 34 Calcium lake, D&C Red No. 36 lake, D&C Red No. 40 Aluminium lake, D&C Blue No. 1 Aluminium lake, D&C Green No. 3 Aluminium lake, D&C Orange No. 4 Aluminium lake, D&C Orange No. 5 Aluminium lake, D&C Orange No. 5 Zirconium lake, D&C Orange No. 10 Aluminium lake, D&C Orange No. 17 Barium lake, D&C Yellow No. 5 Aluminium lake, D&C Yellow No. 5 Zirconium lake, D&C Yellow No. 6 Aluminium lake, D&C Yellow No. 7 Zirconium lake, D&C Yellow No. 10 Aluminium lake, FD&C Blue No. 1 Aluminium lake, FD&C Red No. 4 Aluminium lake, FD&C Red No. 40 Aluminium lake, FD&C Yellow No. 5 Aluminium lake and FD&C Yellow No. 6 Aluminium lake.

The pigments may also have been subjected to a hydrophobic treatment.

The hydrophobic treatment agent may be chosen from silicones such as methicones, dimethicones, alkoxysilanes and perfluoroalkylsilanes; fatty acids such as stearic acid; metal soaps such as aluminium dimyristate, the aluminium salt of hydrogenated tallow glutamate, perfluoroalkyl phosphates, perfluoroalkylsilanes, perfluoroalkylsilazanes, polyhexafluoropropylene oxides, polyorganosiloxanes comprising perfluoroalkyl perfluoropolyether groups, and amino acids; N-acylamino acids or salts thereof; lecithin, isopropyl triisostearyl titanate, and mixtures thereof.

The N-acylamino acids can comprise an acyl group containing from 8 to 22 carbon atoms, such as, for example, a 2-ethylhexanoyl, caproyl, lauroyl, myristoyl, palmitoyl, stearoyl or cocoyl group. The salts of these compounds may be aluminium, magnesium, calcium, zirconium, zinc, sodium or potassium salts. The amino acid may be, for example, lysine, glutamic acid or alanine.

The term “alkyl” mentioned in the compounds cited above in particular denotes an alkyl group containing from 1 to 30 carbon atoms and preferably containing from 5 to 16 carbon atoms.

Hydrophobically treated pigments are described in particular in patent application EP-A-1 086 683.

### Liposoluble Dyes

Liposoluble dyes, such as, one soluble in the medium of the composition, can be useful. Examples can be made to Sudan Red, DC Red 17, DC Green 6, β-carotene, soybean oil, Sudan Brown, DC Yellow 11, DC Violet 2, DC Orange 5 and quinoline yellow.

### Nacres

For the purposes of the present patent application, the term “nacre” means coloured particles of any shape, which may or may not be iridescent, in particular produced by certain molluscs in their shell, or alternatively synthesized, and which have a colour effect via optical interference.

Examples of nacres that may be mentioned include nacreous pigments such as titanium mica coated with an iron oxide, mica coated with bismuth oxychloride, titanium mica coated with chromium oxide, titanium mica coated with an organic dye in particular of the abovementioned type, and also nacreous pigments based on bismuth oxychloride.

They may also be mica particles, at the surface of which are superposed at least two successive layers of metal oxides and/or of organic colorants.

The nacres may more particularly have a yellow, pink, red, bronze, orangey, brown, gold and/or coppery colour or tint.

As illustrations of nacres that may be introduced as interference pigments into the first composition, mention may be made of the gold-coloured nacres sold in particular by the company BASF under the name Brilliant gold 212G (Timica), Gold 222C (Cloisonne), Sparkle gold (Timica) and Monarch gold 233X (Cloisonne); the bronze nacres sold in particular by the company Merck under the name Bronze fine (17384) (Colorona) and Bronze (17353) (Colorona) and by the company BASF under the name Super bronze (Cloisonne); the orange nacres sold in particular by the company BASF under the name Orange 363C (Cloisonne) and by the company Merck under the name Passion orange (Colorona) and Matte orange (17449) (Microna); the brown tinted nacres sold in particular by the company Engelhard under the name Nu-antique copper 340XB (Cloisonne) and Brown CL4509 (Chroma-lite); the copper-tinted nacres sold in particular by the company BASF under the name Copper 340A (Timica); the red-tinted nacres sold in particular by the company Merck under the name Sienna fine (17386) (Colorona); the yellow-tinted nacres sold in particular by the company BASF under the name Yellow (4502) (Chromalite); the gold-tinted red nacres sold in particular by the company BASF under the name Sunstone G012 (Gemtone); the pink nacres sold in particular by the company BASF under the name Tan opal G005 (Gemtone); the gold-tinted black nacres sold in particular by the company BASF under the name Nu antique bronze 240 AB (Timica), the blue nacres sold in particular by the company Merck under the name Matte blue (17433) (Microna), the silvery-tinted white nacres sold in particular by the company Merck under the name Xirona Silver, and the golden-green pink-orange nacres sold in particular by the company Merck under the name Indian summer (Xirona), and mixtures thereof.

According to a preferred embodiment, the colorant used in the present invention is chosen from metal oxide pigments, organic lakes, synthetic or natural water-soluble dyes and mixtures thereof.

According to a preferred embodiment, the colorant used in the present invention is selected from titanium dioxides, iron oxides, organic pigments and colorants soluble in the medium of the composition.

According to an exemplary embodiment, the colorant(es) used in the present invention is chosen from titanium dioxide, Yellow 6 lake, Red 7, Blue 1 lake, or mixtures thereof.

The colorant can be present in an amount ranging from 3% to 20% by weight, preferably, 5% to 16% by weight, more preferably 6% to 12% by weight, relative to the total weight of the composition.

### Additives

In a particular embodiment, an anhydrous composition according to the present invention may further comprise at least one additive usually used in the field under consideration. In particular the additive is chosen from gums, anionic, cationic, amphoteric or nonionic surfactants, silicone surfactants, resins, thickening agents, dispersants, antioxidants, preserving agents, fragrances, neutralizers, antiseptics, additional cosmetic active agents, such as vitamins, moisturizers, emollients or collagen-protecting agents, and mixtures thereof.

It is a matter of routine operations for a person skilled in the art to adjust the nature and amount of the additives present in the compositions in accordance with the present invention such that the advantageous properties of the composition used according to the present invention are not, or are not substantially, adversely affected by the envisaged addition.

### Method and Use

The composition according to the present invention can be generally prepared according to the general knowledge of a person skilled in the art. Nevertheless, it is to be understood that a person skilled in the art can choose the method of preparation, on the basis of his/her general knowledge, taking into account the nature of the constituents used, for example, their solubility in the vehicle, and the application envisaged for the compositions or the composition.

According to an embodiment, the composition according to the present invention can be used for treating keratin materials, especially lip. This use may manifest itself as a process for caring for/making up keratin materials, especially the lip, comprising the step applying to said keratin materials the composition of the invention.

The invention will be further illustrated by the following examples, which set forth particularly advantageous embodiments.

Notwithstanding that the numerical ranges and parameters setting forth the broad scope of the present invention are approximations, the numerical values set forth in the specific examples are reported as precisely as possible. Any numerical value, however, inherently contain certain errors necessarily resulting from the standard deviation found in their respective measurements. The following examples are intended to illustrate the present invention without limiting the scope as a result.

### EXAMPLES

The ingredient amounts/concentrations in the compositions/formulas described below were expressed in % by weight, relative to the total weight of each composition/formula.

Main raw materials used, trade names and supplier thereof are listed below. Materials without specification here were each commercially available.

| INCI Name | Trade Name | Supplier |
|---|---|---|
| SQUALANE | NEOSSANCE ® SQUALANE | AMYRIS |
| PENTAERYTHRITYL TETRAISOSTEARATE | JOLEE 7181 | OLEON |
| POLYBUTENE | INDOPOL ® H 100 | INEOS |
| CANOLA OIL | LIPEX ® PREACT ™ | AARHUSKARLSHAMN |
| CAPRYLIC/CAPRIC TRIGLYCERIDE | MASESTER E7000 LR01-1/MB | PT MUSIM MAS |
| POLYETHYLENE | PERFORMALENE ™ 500-L | NEW PHASE |
| | POLYETHYLENE | TECHNOLOGIES |
| PARAFFIN (and) MICROCRYSTALLINE WAX (and) | PARACERA ® 30540 | PARAMELT |
| SYNTHETIC WAX | | |
| HELIANTHUS ANNUUS (SUNFLOWER) SEED WAX | SUNFLOWER WAX DOUBLE | KOSTER KEUNEN |
| | REFINED | |
| RED 7 | UNIPURE ® RED LC 3079 OR | SENSIENT |
| YELLOW 6 LAKE | SUNCROMA ™ FD&C YELLOW 6 | SUN |
| | AL LAKE C70-5270 | |
| RED 28 LAKE | SUNCROMA ™ D&C RED 28 AL | SUN |
| | LAKE C14-6623 | |
| DIISOSTEAROYL POLYGLYCERYL-3 DIMER | SOLAMAZE ™ NATURAL | AKZO NOBEL |
| DILINOLEATE (and) CAPRYLIC/CAPRIC | | (NOURYON) |
| TRIGLYCERIDE | | |
| C30-45 ALKYLDIMETHYLSILYL | DOW CORNING ® SW-8005 C30 | DOW CORNING |
| POLYPROPYLSILSESQUIOXANE | RESIN WAX | (DOW CHEMICAL) |
| VP/HEXADECENE COPOLYMER | ANTARON ™ V 216 polymer OU | ISP (ASHLAND) |
| | GANEX V 216 | |
| ALLYL STEARATE/VA COPOLYMER | MEXOMERE PQ | NOVEAL |
| HYDROGENATED CASTOR OIL/SEBACIC ACID | CRODABOND ™ CSA-LQ-(JP) | CRODA |
| COPOLYMER | | |
| DILINOLEIC ACID/BUTANEDIOL COPOLYMER | VISCOPLAST 14436 H | BIOSYNTHIS |
| VP/EICOSENE COPOLYMER | ANTARON ™ V 220F polymer OU | ISP (ASHLAND) |
| | GANEX ™ V 220F | |
| HYDROGENATED | REGALITE ™ R1100 CG | EASTMAN |
| STYRENE/METHYLSTYRENE/INDENE | HYDROCARBON RESIN | CHEMICAL |
| COPOLYMER | | |

### Protocol of Preparation

The lipsticks were prepared following the steps of:
1). milling pigments (Red 7, Red 28 Lake, and/or Yellow 6 Lake) with the oil using a three-roll miller;
2). weighing and adding the fatty phase (oils, waxes, surfactants, pastes and/or polymers) into a main vessel and heating the vessel to around 95° C. with stirring until a homogeneous mixture was achieved;
3). adding the glycerin into the main vessel and homogenizing the bulk at a strong agitation speed of 1500 rpm;
4). adding the pigment paste prepared in step 1);
5). once the bulk was uniform, doing lipstick molding, cooling, and picking.

### Example A

For different purposes, Ex.1-Ex.2 according to the present invention, as well as comparative CE.1-CE.8

TABLE 1

| Components | CE. 1 | Ex. 1 | Ex. 2 | CE. 2 | CE. 3 | CE. 4 | CE. 5 | CE. 6 | CE. 7 | CE. 8 |
|---|---|---|---|---|---|---|---|---|---|---|
| SQUALANE | QS to 100 | | | | | | | | | |
| PENTAERYTHRITYL | 10 | 10 | 10 | 10 | 10 | 10 | 10 | 10 | 10 | 10 |
| TETRAISOSTEARATE | | | | | | | | | | |
| POLYBUTENE | 4 | 4 | 4 | 4 | 4 | 4 | 4 | 4 | 4 | 4 |
| POLYETHYLENE | 10 | 10 | 10 | 10 | 10 | 10 | 10 | 10 | 10 | 10 |
| PARAFFIN (and) | 1 | 1 | 1 | 1 | 1 | 1 | 1 | 1 | 1 | 1 |
| MICROCRYSTALLINE WAX (and) | | | | | | | | | | |
| SYNTHETIC WAX | | | | | | | | | | |
| DIISOSTEAROYL | — | 4 | 10 | — | — | — | — | — | — | — |
| POLYGLYCERYL-3 DIMER | | | | | | | | | | |
| DILINOLEATE (and) CAPRYLIC/ | | | | | | | | | | |
| CAPRIC TRIGLYCERIDE | | | | | | | | | | |
| RED 7 | 3 | 3 | 3 | 3 | 3 | 3 | 3 | 3 | 3 | 3 |
| YELLOW 6 LAKE | 5 | 5 | 5 | 5 | 5 | 5 | 5 | 5 | 5 | 5 |
| RED 28 LAKE | 2 | 2 | 2 | 2 | 2 | 2 | 2 | 2 | 2 | 2 |
| C30-45 ALKYLDIMETHYLSILYL | — | — | — | 4 | — | — | — | — | — | — |
| POLYPROPYLSILSESQUIOXANE | | | | | | | | | | |
| VP/HEXADECENE COPOLYMER | — | — | — | — | 4 | — | — | — | — | — |
| ALLYL STEARATE/VA COPOLYMER | — | — | — | — | — | 4 | — | — | — | — |
| HYDROGENATED CASTOR | — | — | — | — | — | — | 4 | — | — | — |
| OIL/SEBACIC ACID COPOLYMER | | | | | | | | | | |
| DILINOLEIC ACID/BUTANEDIOL | — | — | — | — | — | — | — | 4 | — | — |
| COPOLYMER | | | | | | | | | | |
| VP/EICOSENE COPOLYMER | — | — | — | — | — | — | — | — | 4 | — |
| HYDROGENATED | — | — | — | — | — | — | — | — | — | 4 |
| STYRENE/METHYLSTYRENE/INDENE | | | | | | | | | | |
| COPOLYMER | | | | | | | | | | |

### Example B

The compositions of Example A were evaluated for the wearing/non-transfer performance according to the following steps:
applying the composition on forearm 3 times;
staying for 45 min;
using a tissue to cover the application area and pressing with the constant force for 10s;
scoring 1˜5 points according to the color intensity imprinted on the tissue: the lower the score, the lower the color intensity on the tissue, while the better non-transfer performance; and
averaging the scores of evaluations done by at least 3 experienced scientists.

The scores were provided in Table 2 below:

TABLE 2

| Formulas | Score of Transfer Test |
|---|---|
| CE. 1 | 5 |
| Ex. 1 | 2.8 |
| Ex. 2 | 1 |
| CE. 2 | 4 |
| CE. 3 | 4.8 |
| CE. 4 | 4.5 |
| CE. 5 | 4 |
| CE. 6 | 4.5 |
| CE. 7 | 5 |
| CE. 8 | 5 |

A part of the compositions of Example A were further evaluated and scored for the wearing/non-transfer performance by a consumer panel according to the following steps:
selecting 12 women aged 18˜40 years old;
asking 12 users to try one composition on lip per day; and
collecting their feedbacks after they tested all the provided compositions.

The scores were provided in Table 3 below:

TABLE 3

| Number of | Comments on wearing performance |
|---|---|
| consumers | (“>” means “better than”, “=” means “close to”) |
| 7 | Ex. 2 > Ex. 1 > CE. 1 |
| 3 | Ex. 2 = Ex. 1 > CE. 1 |

### Example C

Following Ex.1, additional Ex.3 and Ex.4 were prepared as follows:

TABLE 4

| Components | Ex. 1 | Ex. 3 | Ex. 4 |
|---|---|---|---|
| PENTAERYTHRITYL | 10 | 20 | 10 |
| TETRAISOSTEARATE | | | |
| POLYBUTENE | 4 | — | 4 |
| CAPRYLIC/CAPRIC | — | 5 | — |
| TRIGLYCERIDE | | | |
| POLYETHYLENE | 10 | 10 | 7 |
| PARAFFIN (and) | 1 | 1 | 0 |
| MICROCRYSTALLINE WAX (and) | | | |
| SYNTHETIC WAX | | | |
| HELIANTHUS ANNUUS | — | — | 2 |
| (SUNFLOWER) SEED WAX | | | |
| EUPHORBIA CERIFERA | — | — | 2 |
| (CANDELILLA) WAX | | | |
| DIISOSTEAROYL | 4 | 4 | 4 |
| POLYGLYCERYL-3 DIMER | | | |
| DILINOLEATE (and) CAPRYLIC/ | | | |
| CAPRIC TRIGLYCERIDE | | | |
| RED 7 | 3 | 3 | 3 |
| YELLOW 6 LAKE | 5 | 5 | 5 |
| RED 28 LAKE | 2 | 2 | 2 |
| SQUALANE | QS to 100 | 10 | QS to 100 |
| CANOLA OIL | — | QS to 100 | — |

The compositions of Ex.1 to Ex.4 were evaluated for the wearing/non-transfer performance according to Example B. The results for all of Ex. I to Ex.4 were summarized below:

TABLE 5

| Formulas | Score of Transfer Test |
|---|---|
| Ex. 1 | 2.8 |
| Ex. 2 | 1 |
| Ex. 3 | 1.5 |
| Ex. 4 | 1 |

It could be seen that using the composition of the invention, especially appropriately amount of DIISOSTEAROYL POLYGLYCERYL-3 DIMER DILINOLEATE (and) CAPRYLIC/CAPRIC TRIGLYCERIDE, desirable non-transfer performances were achieved.

1. An anhydrous composition for making up keratin materials, comprising:
a mixture of
a) a polyester which is the reaction product of a polyglycerol-3; a dimer acid; and a C₈-C₃₀ fatty monoacid, in a mole ratio of 1 mole polyglycerol-3, 0.5 to 1 mol dimer acid, and from 0.1 to less than 2.0 mole fatty acids; and
b) a non-volatile non-silicone oil H1;
a wax;
at least one colorant selected from the group consisting of titanium dioxides, iron oxides, organic pigments and colorants soluble in the medium of the composition; and optionally
a non-volatile non-silicone oil H2.

2. The composition according to claim 1, wherein the polyglycerol-3 is triglycerol alone or a mixture of polyglycerols comprising at least triglycerol.

3. The composition according to claim 2, wherein the polyglycerol-3 is a mixture of polyglycerols comprising triglycerol and polyglycerols which are oligocondensation products of glycerol of formula (I):
H[—O-Gly-]ₙ-OH (I)
wherein each Gly is independently the residue of a molecule of glycerol after removal of two hydroxyl groups; and n is an average of from 2 to 10.

4. The composition according to claim 2, wherein the polyglycerol-3 is a mixture comprising oligoglycerols having the oligomer distribution of, based on a total content of the polyglycerols:
Glycerol: 0 to 30% by weight,
Diglycerol: 10 to 40% by weight,
Triglycerol: 10 to 65% by weight,
Tetraglycerol: 2 to 25% by weight,
Pentaglycerol: 0 to 15% by weight,
Hexaglycerol: 0 to 15% by weight,
Heptaglycerol: 0 to 10% by weight,
Octaglycerol: 0 to 10% by weight,
Nonaglycerol: 0 to 5% by weight, and
Decaglycerol: 0 to 5% by weight, relative to the total weight of the polyglycerol-3.

5. The composition according to claim 1, wherein the polyglycerol-3 comprises a mixture of oligoglycerols having an oligomer distribution of, based on a total content of the polyglycerols:
Glycerol: 0 to 30% by weight
Diglycerol: 15 to 40% by weight
Triglycerol: 10 to 55% by weight
Tetraglycerol: 2 to 25% by weight
Pentaglycerol and higher components: 0 to 15% by weight relative to the total weight of the polyglycerol-3.

6. The composition according to claim 5, wherein the polyglycerol-3 mixture comprises at least 40% by weight, based on a total weight of the polyglycerol-3 of a combination of diglycerol and triglycerol.

7. The composition according to claim 5, wherein the polyglycerol-3 mixture comprises at least 20% by weight of diglycerol; at least 15% by weight of triglycerol; and at least 10% by weight by weight of tetraglycerol; wherein all weight percentages are based on a total content of the polyglycerol-3 mixture.

8. The composition according to claim 1, wherein the dimer acid is a hydrogenated dimer acid containing from 60% by weight to 95% by weight of hydrogenated C₃₆ dimer acid and from 5% to 30% by weight of C₅₄ hydrogenated trimer acids.

9. The composition according to claim 1, wherein the C₈-C₃₀ fatty monoacid is selected from the group consisting of Caprylic acid (C₈), Pelargonic acid (C₉), Capric acid (C₁₀), Undecylic acid (C₁₁), Lauric acid (C₁₂), Tridecylic acid (C₁₃), Myristic acid (C₁₄), Pentadecylic acid (C₁₅), Palmitic acid (C₁₆), Margaric acid (C₁₇), Stearic acid (C₁₈), Isostearic acid (C₁₈), Nonadecylic acid (C₁₉), Arachidic acid (C₂₀), Behenic acid (C₂₂), and Lignoceric acid (C₂₄).

10. The composition according to claim 1, wherein the polyester is a substantially or totally non sequential reaction product of the following components:
i) a polyglycerol-3 mixture comprising at least 25% by weight diglycerol, at least 45% by weight triglycerol, and at least 10% by weight tetraglycerol, in each case based on a total weight of the polyglycerol-3 mixture;
ii) a hydrogenated dimer acid containing at least 60% by weight of hydrogenated C₃₆ diacid and 5-25% by weight of hydrogenated C₅₄ triacid, in each case based on a total weight of hydrogenated acid; and
iii) isostearic acid.

11. The composition according to claim 1, wherein the mole ratio of the polyester reaction product is 1 mole polyglycerol, 0.5 to 1 mol dimer acid and 0.2 to 1.7 mole fatty acid; or 1 mole polyglycerol-3, 0.5 to 0.75 mol dimer acid, and 0.4 to 1.35 mole isostearic acid; or 1 mole polyglycerol-3, 0.5 to 0.7 mol dimer acid, and 0.65 to 1 mole isostearic acid.

12. The composition according to claim 1, wherein the polyester has a weight-average molecular weight of from 5000 Da to 15,000 Da measured with GPC using linear polystyrene standards and a viscosity of the neat polyester is from 100,000 mPa·s to 2,000,000 mPa·s at 25° C.

13. The composition according to claim 1, wherein a total esterification of the polyester is from 28% to 57% by weight, including an esterification with dimer acid of 20% to 30% by weight and an esterification with monoacid between 8% and 27% by weight.

14. The composition according to claim 1, wherein the non-volatile non-silicone oil H1 or the non-volatile non-silicone oil H2 is each independently selected from the group consisting of fatty acid triglycerides containing from 4 to 24 carbon atoms, polybutenes, polyisobutenes, hydrogenated polyisobutenes, polydecenes, hydrogenated polydecenes, squalene, esters of C₄-C₂₂ dicarboxylic or tricarboxylic acids and of C₁-C₂₂ alcohols and esters of mono-, di- or tri-carboxylic acids and of C₂-C₂₆ di-, tri-, tetra- or penta-hydroxy alcohols, and mixtures thereof.

15. The composition according to claim 1, wherein the mixture is present in the anhydrous composition in an amount of from 0.01% to 20% by weight based on the total weight of the total anhydrous composition.

16. The composition according to claim 1, wherein the wax is selected from the group consisting of hydrocarbon-based waxes, for instance beeswax, lanolin wax or Chinese insect wax; rice wax, carnauba wax, candelilla wax, euphorbia cerifera (candelilla) wax, ouricury wax, esparto grass wax, cork fibre wax, sugar cane wax, Japan wax, sumach wax, Helianthus annuus (sunflower) seed wax; montan wax, microcrystalline waxes, paraffins and ozokerite; polyethylene waxes, polymethylene waxes; synthetic wax, paraffin, microcrystalline wax, or mixtures thereof.

17. The composition according to claim 1, wherein an amount of the wax(es) is from 0.01% to 20% by weight, relative to the total weight of the anhydrous composition.

18. A method for making up keratin materials, comprising applying the composition of claim 1 to the keratin material, wherein the keratin material is skin or lips.
